(19) United States
(12) Patent Application Publication
(10) Pub. No.: US 2007/0258853 A1
(43) Pub. Date: Nov. 8, 2007

(54) Biocidal Treatment Device

(76) Inventor: John Burton, Osborne Park WA (AU)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/663,149

(22) PCT Filed: Sep. 16, 2005
(86) PCT No.: PCT/AU05/01427

§ 371 Date: Mar. 16, 2007

(30) Foreign Application Priority Data
Sep. 17, 2004 (AU) 2004905361

(51) Int. Cl. A61L 2/07

(52) U.S. Cl. 422/26

(57) ABSTRACT

A biocidal treatment device is disclosed which comprises at least one gas water heater arranged to produce heated water for use in biocidal treatment, connection means for facilitating connection to at least one gaseous fuel source arranged to store and supply gaseous fuel during use to the at least one gas water heater, at least one gas conduit extending between the connection means and the at least one gas water heater, and a liquid reservoir. A portion of the at least one gas conduit extends through the liquid reservoir so as to facilitate transfer of heat to the at least one gas conduit during use and thereby restrict freezing of the gas conduit during use. The liquid reservoir may be a water reservoir arranged to store and supply water to the at least one gas water heater.

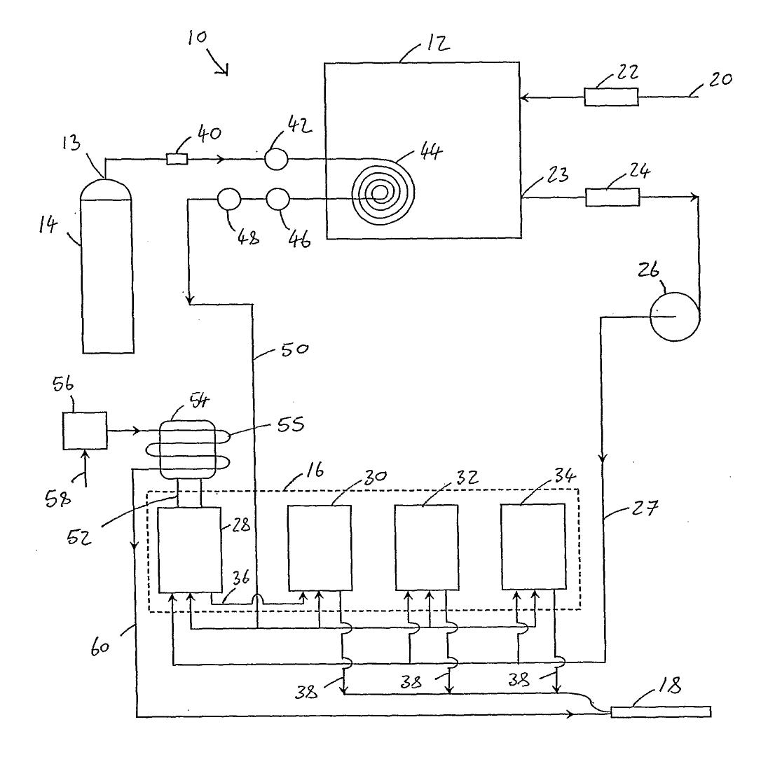

### FIELD OF THE INVENTION

The present invention relates to a biocidal treatment device of the type which is capable of generating and supplying heated water to a hand held wand and thereby to a region containing plant vegetation, pests, and so on for the purpose of biocidally treating the region.

### BACKGROUND OF THE INVENTION

It is known to carry out biocidal treatment of plant vegetation and pests by generating and supplying pressurised superheated water to the plant vegetation and/or pests, such pressurised superheated water being generated in one example using a diesel water heater.

However, diesel fuel is relatively messy because diesel has a relatively low volatility.

### SUMMARY OF THE INVENTION

In accordance with a first aspect of the present invention, there is provided a biocidal treatment device comprising:
at least one gas water heater arranged to produce heated water for use in biocidal treatment;
connection means for facilitating connection to at least one gaseous fuel source arranged to store and supply gaseous fuel during use to the at least one gas water heater;
at least one gas conduit extending between the connection means and the at least one gas water heater; and
a liquid reservoir;
a portion of the at least one gas conduit extending through the liquid reservoir so as to facilitate transfer of heat to the at least one gas conduit during use and thereby restrict freezing of the gas conduit during use.

In one arrangement, the liquid reservoir is a water reservoir arranged to store and supply water to the at least one gas water heater. In an alternative arrangement, the liquid reservoir is a first water reservoir and the device further includes a second water reservoir for storing and supplying water to the at least one gas water heater.

In one embodiment, the at least one gas water heater includes at least one primary water heater arranged to pre-heat water, and at least one secondary water heater arranged to superheat water. One primary water heater and three secondary water heaters may be provided, and pre-heated water produced by the primary water heater may be supplied to one or more of the secondary water heaters.

At least one of the water heaters may be arranged such that an exhaust of the water heater is used to pre-heat compressed air used to expel superheated water during use. For this purpose, the water heater may be provided with heat exchanging means arranged to transfer heat from exhaust gases to the compressed air. In one embodiment, the heat exchanging means takes the form of at least one heat exchanging pipe, the at least one heat exchanging pipe containing the compressed air and extending through the exhaust gases during use.

In one arrangement, the portion of the at least one gas conduit extending through the liquid reservoir is configured in the form of a coil.

The connection means may facilitate connection to an LPG gaseous fuel source.

### BRIEF DESCRIPTION OF THE DRAWING

The present invention will now be described with reference to the accompanying drawing which is a schematic diagram of a biocidal treatment device in accordance with an embodiment of the present invention.

### DESCRIPTION OF AN EMBODIMENT OF THE INVENTION

Referring to the drawing, there is shown a biocidal treatment device 10 of the type arranged to generate superheated water and supply the superheated water under pressure to a region so that the region can be biocidally treated.

The biocidal treatment device 10 includes a water tank 12 for storing water to be superheated by the device 10, water heating means 16 arranged to superheat the water supplied by the water tank 12, and a wand 18 which is held during use by an operator and which facilitates directability of the superheated water and thereby user selection of the region which receives superheated water during use.

The device is connectible at connection means 13 to a gaseous fuel cylinder 14, in this example a liquid petroleum gas (LPG) cylinder.

The water tank 12 is capable of receiving fresh water through a water inlet 20, an inlet filter 22 being disposed between the water inlet 20 and the water tank 12 to filter the fresh water as it passes to the water tank 12. The water tank 12 in this example has a capacity of the order of 1600 litres and includes a water outlet 23 through which water passes during use, water from the water outlet 23 passing through an outlet filter 24 and being pumped by a water pump 26 through a water conduit 27 to the water heating means 16 during use. In this example, the pump 26 is capable of pumping up to 32 litres per minute of water at 81 psi, although it will be understood that other pumps are envisaged, the important aspect being that the pump is capable of supplying a sufficient amount of water for heating by the water heating means 16 and supply to the wand 18.

The water heating means 16 in this example includes first, second, third and fourth water heaters 28, 30, 32 and 34 respectively, the first water heater 28 serving as a pre-heater for part of the water, the second water heater 30 serving to further heat the water pre-heated by the first water heater 28, and the third and fourth water heaters serving to heat water without pre-heating.

In this example the first water heater 28 heats water to about 75° C. to 80° C., and the second, third and fourth water heaters heat the water to about 100° C. to 115° C.

Superheated water produced by the second, third and fourth water heaters 30, 32, 34 passes through superheated water conduits 38 to the wand 18 and thereby under control of an operator to a biocidal treatment region.

The gas cylinder 14 contains liquefied gaseous fuel, in this example liquefied LPG. Flow of LPG from the gas cylinder 14 is controllable using a valve, in this example a solenoid valve 40. When the solenoid valve 40 is open, liquefied LPG passes into a first pressure regulator 42 which in this example serves to drop the pressure of the liquefied LPG to about 30 psi. The LPG then passes into a first heat exchanging means, in this example in the form of a coil 44 formed of suitable heat conductive tubing.

In this example, the coil 44 is formed of copper tubing approximately 18 m long and approximately 1.27 cm in diameter, and the first heat exchanging means 44 is disposed in the water tank 12 so as to facilitate exchange of heat from water in the water tank 12, through the coil and to the LPG inside the coil. In this way, by providing a sufficient length of heat conductive tubing inside the water tank 12, it is possible to avoid freezing of the coil 44 due to the rapid drop in pressure of the LPG.

Although in this example the first heat exchanging means 44 is in the form of a coil, it will be understood that other arrangements are possible, the important aspect being that a sufficient amount of tubing is disposed inside the water tank 12 to prevent freezing.

Although the first heat exchanging means 44 is disposed inside the water tank 12, it will be understood that as an alternative a separate liquid container may be provided solely for the purpose of receiving the first heat exchanging means and transferring heat to the heat conductive tubing to prevent freezing.

After passing through the first heat-exchanging means 44, the LPG passes through a second pressure regulator 46 which drops the pressure of the LPG from about 30 psi to about 15 psi. The LPG then passes into a third pressure regulator 48 which drops the pressure of the LPG from about 15 psi to about 3.5 psi. The third pressure regulator 48 in this example also includes an over pressure shut-off (OPSO) device which serves as a safety measure in the event of excessive pressure from the gas cylinder 14. The LPG then passes through a gas conduit 50 which feeds the now gaseous LPG to the first, second, third and fourth water heaters 28, 30, 32, 34.

In addition to carrying out the function of pre-heating a portion of water, the first water heater 28 also serves to pre-heat air which is supplied to the wand 18. For this purpose, an exhaust 52 of the first water heater 28 is connected to a second heat exchanging means 54, in this example in the form of a jacket having heat-exchanging pipes 55 passing therethrough, in this example the heat exchanging pipes 55 being formed of copper tubing.

Air is supplied to the pipes 55 using a compressor 56 which serves to receive air through an air inlet 58 and supply pressurised air to the pipes 55 during use. Pressurised heated air from the pipes 55 is then supplied through an air conduit 60 to the wand 18. In this example, the heated air from the heat exchanging pipes 55 is at a temperature of the order of 100° C. to 350° C.

During use, an operator holds the wand 18 and points the wand 18 at a region desired to be biocidally treated. On activation of the device 10 using appropriate control means such as switches (not shown) to activate the pump 26, the first, second, third and fourth heaters 28, 30, 32, 34 and the solenoid 40, water from the tank 12 is pumped to the first water heater 28 for pre-heating and to the second, third and fourth water heaters 30, 32, 34 for superheating, the superheated water then flowing to the wand 18. At the same time, air is pressurised by the compressor 56, pre-heated by the second heat-exchanging means 54 and supplied to the wand 18. The compressed air and superheated water combine in the wand 18 and, under appropriate control of an operator, cause ejection of superheated water from the wand 18 towards the desired biocidal treatment region.

It will be understood that since the compressed air is pre-heated, the superheated water ejected from the wand by the compressed air is maintained at a temperature of the order of 100° C. to 115° C., thereby enhancing the efficiency of the biocidal treatment.

It will also be understood that by providing the first heat exchanging means, it becomes practically possible to use liquefied gaseous fuel, such as LPG, as a fuel source since freezing of the pipes carrying the LPG as the pressure of the LPG drops is prevented.

Modifications and variations as would be apparent to a skilled addressee are deemed to be within the scope of the present invention.

1.-10. (canceled)

11. A biocidal treatment device comprising:
at least one gas water heater arranged to produce heated water for use in biocidal treatment;
a water reservoir arranged to store water and supply water to the at least one gas water heater;
means for directing heated water produced by the at least one gas water heater towards a desired location;
connection means for facilitating connection to at least one gaseous fuel source arranged to store and supply gaseous fuel during use to the at least one gas water heater; and
at least one gas conduit extending between the connection means and the at least one gas water heater; and
a portion of the at least one gas conduit extending through the water reservoir so as to facilitate transfer of heat to the at least one gas conduit during use and thereby restrict freezing of the gas conduit during use.

12. A device as claimed in claim 11, wherein the at least one gas water heater includes at least one primary water heater arranged to pre-heat water, and at least one secondary water heater arranged to superheat water.

13. A device as claimed in claim 12, wherein one primary water heater and three secondary water heaters are provided, pre-heated water produced by the primary water heater being supplied to one or more of the secondary water heaters.

14. A device as claimed in claim 11, wherein at least one water heater is arranged such that an exhaust of the water heater is used to pre-heat compressed air used to expel superheated water during use.

15. A device as claimed in claim 14, wherein the at least one water heater is provided with heat exchanging means arranged to transfer heat from exhaust gases to the compressed air, the at least one heat exchanging means including at least one heat exchanging pipe containing the compressed air and extending through the exhaust gases during use.

16. A device as claimed in claim I 1, wherein the portion of the at least one gas conduit extending through the liquid reservoir is configured in the form of a coil.

17. A device as claimed in claim I1, wherein the connection means facilitates connection to an LPG gaseous fuel source.
